(19) United States
(12) Patent Application Publication
(10) Pub. No.: US 2018/0326176 A1
(43) Pub. Date: Nov. 15, 2018

(54) DEVICE FOR SIMULATING A HUMAN PRESENCE AND METHOD

(71) Applicant: NEIL CLOET, SUTTON, NE (US)

(72) Inventor: NEIL CLOET, SUTTON, NE (US)

(21) Appl. No.: 15/595,091

(22) Filed: May 15, 2017

(51) Int. Cl. A61M 21/02; A47D 7/00

(52) U.S. Cl. CPC A61M 21/02; A61M 2021/0061; A47D 7/00

(57) ABSTRACT

A device for simulating a human presence and method is disclosed herein. The device includes a speaker configured to provide an audio output substantially similar to a voice of the remote person, a heartbeat simulator configured to provide a tactile output onto a bed substantially similar to a heartbeat of the remote person, a processor communicably coupled to the speaker and to the heartbeat simulator, the processor configured to drive the speaker and the heartbeat simulator, and a power supply electrically coupled to the speaker and to the heartbeat simulator, the power supply configured to power the speaker and the heartbeat simulator. The device for simulating a human presence is useful for mimicking the heartbeat of a mother to comfort a baby or child and maximize sleep time.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. FIELD OF THE INVENTION

The present disclosure relates generally to the field of beds and more specifically relates to mattresses and accessories.

### 2. DESCRIPTION OF RELATED ART

Many times infants can have trouble sleeping at night or during nap time. This can often be caused by separation anxiety from being inside the womb with the mother to outside the womb. Inside the womb, the unborn child receives audio stimulation. Such stimulation may include the mother's heartbeat, gastro-intestinal sounds, the mother's voice, as well as sounds external to the womb such as music of the voices of others. Often times, a white noise machine can be used to provide audio sounds to an infant or other child.

A white noise machine is a device that produces a sound that is random in character, which sounds like a rushing waterfall or wind blowing through trees. Often such devices do not produce actual white noise, which has a harsh sound, but pink noise, whose power rolls off at higher frequencies, or other colors of noise.

U.S. Pat. No. 4,947,832 to Avrum H. Blitzer relates to an apparatus and method for treating colicky infants. The described apparatus includes a device to mechanically simulate womb conditions to ease the transition from womb to post-gestational life. The device provides a regular, repetitive low frequency sine wave motion progressively traveling longitudinally thereof with simultaneous vibrations, whereby the infant experiences gentle, low frequency, regular, progressive, traveling sine wave motion plus hearing and sensing subdued vibrations like sensations previously experienced in the womb. The device includes an upper, mattress-supporting layer having a longitudinal series of transverse slats, gently moved up and down by motor-driven cams providing a distributed drive producing regular, repetitive, progressively traveling sine wave motion simulating peristalsis effects of an intestine near a uterine wall. A one-inch thick foam mattress covering the slats provides the longitudinally traveling sine wave motion undulating up and down.

However, such devices fail to effectively mimic the mother's heartbeat or provide voice-audio capabilities. Therefore a suitable solution is desired.

### BRIEF SUMMARY OF THE INVENTION

In view of the foregoing disadvantages inherent in the known bed art, the present disclosure provides a novel recordable mattress device. The general purpose of the present disclosure, which will be described subsequently in greater detail, is to provide a device that is incorporated into a mattress to allow the recording and/or playback of sounds.

A device for simulating a human presence of a remote person is disclosed herein. The device includes a speaker configured to provide an audio output substantially similar to a voice of the remote person, a heartbeat simulator configured to provide a tactile output onto a bed substantially similar to a heartbeat of the remote person, a processor communicably coupled to the speaker and to the heartbeat simulator, the processor configured to drive the speaker and the heartbeat simulator, and a power supply electrically coupled to the speaker and to the heartbeat simulator, the power supply configured to power the speaker and the heartbeat simulator.

According to one embodiment, the device for simulating a human presence of a remote person may be removable from a mattress. Additionally, the device for simulating a human presence of a remote person may include a mattress assembly into which the device for simulating a human presence of a remote person may be placed.

According to another embodiment, a method of using a recordable mattress device is also disclosed herein. The method of using a recordable mattress device includes a first step, providing the recordable mattress device and a mattress assembly; a second step, recording a first audio track on the recordable mattress device; a third step, inserting the recordable mattress device into the mattress; a fourth step, placing a child upon the mattress assembly; a fifth step, reproducing the audio track such that the child can hear the audio track; a sixth step, removing the child from the mattress assembly; and a seventh step, recording a second audio track on the recordable mattress device. It should be noted that not all steps need be implemented in all cases, nor the steps need be performed in a certain order.

For purposes of summarizing the disclosure, certain aspects, advantages, and novel features of the disclosure have been described herein. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE DRAWINGS

The figures which accompany the written portion of this specification illustrate embodiments and methods of use for the present disclosure, a device for simulating a human presence and method, constructed and operative according to the teachings of the present disclosure.

FIG. 1 is a perspective view of the device for simulating a human presence during an ‘in-use’ condition, according to an embodiment of the disclosure.

FIG. 2 is a diagram of the device for simulating a human presence of FIG. 1, showing the components, according to an embodiment of the present disclosure.

FIG. 3 is a perspective view of the device for simulating a human presence including a mattress assembly of FIG. 1, according to an embodiment of the present disclosure.

FIG. 4 is an exploded view of the device for simulating a human presence of FIG. 1, showing the speaker, the heartbeat simulator, the processor, and the power supply as removable from the mattress assembly according to an embodiment of the present disclosure.

FIG. 5 is a flow diagram illustrating a method of use for a device for simulating a human presence, according to an embodiment of the present disclosure.

The various embodiments of the present disclosure will hereinafter be described in conjunction with the appended drawings, wherein like designations denote like elements.

### DETAILED DESCRIPTION

As discussed above, embodiments of the present disclosure relate to mattresses and accessories and more particularly to a device for simulating a human presence and method as used to improve the soothing of an infant or child by providing physical and/or auditory stimulation which may resemble a human presence.

Generally, a device for simulating a human presence may be useful for mimicking the heartbeat of a mother to comfort a baby or child, and may increase sleep time. The device may include a means for creating an electronic heartbeat to comfort a baby and promote sleeping. The present disclosure may be used by users of all ages. The sound of a heartbeat is naturally soothing and comforting to a user; the device may be used in hospitals, at home, or other places for increased comfort of the user.

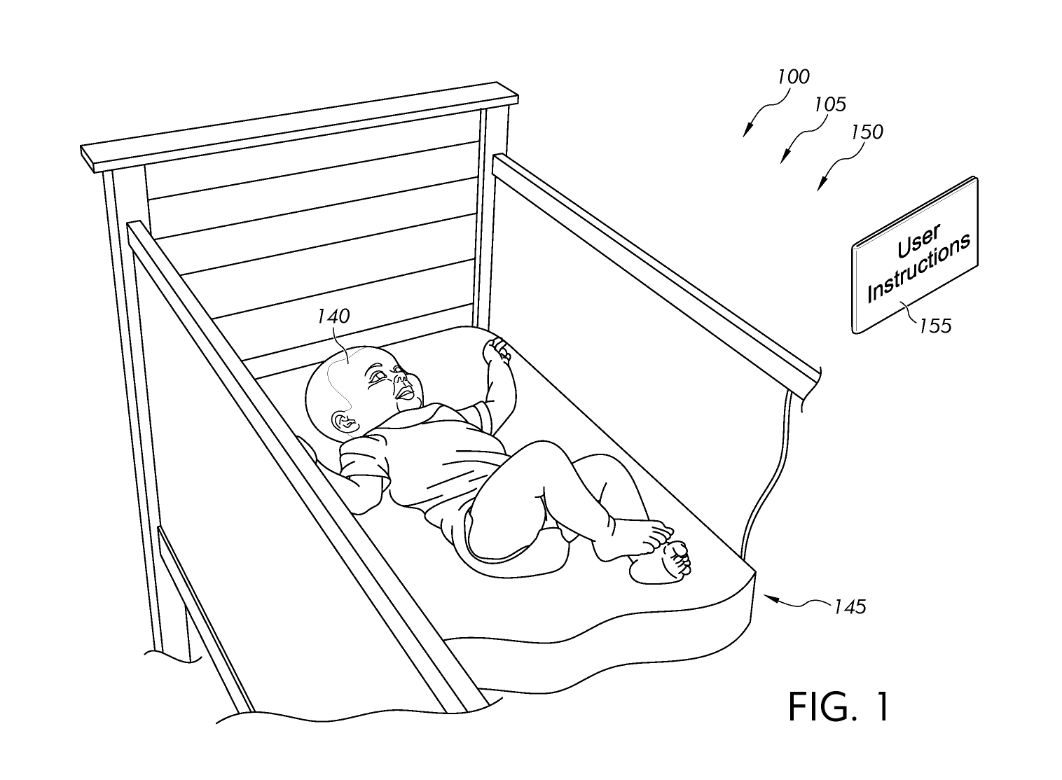

Referring now more specifically to the drawings by numerals of reference, there is shown in FIGS. 1-4, various views of a device for simulating a human presence 100. FIG. 1 shows a device for simulating a human presence 100 during an ‘in-use’ condition 150, according to an embodiment of the present disclosure. Here, the device for simulating a human presence 100 may be beneficial for use by a user 140 to provide a soothing effect and comfort.

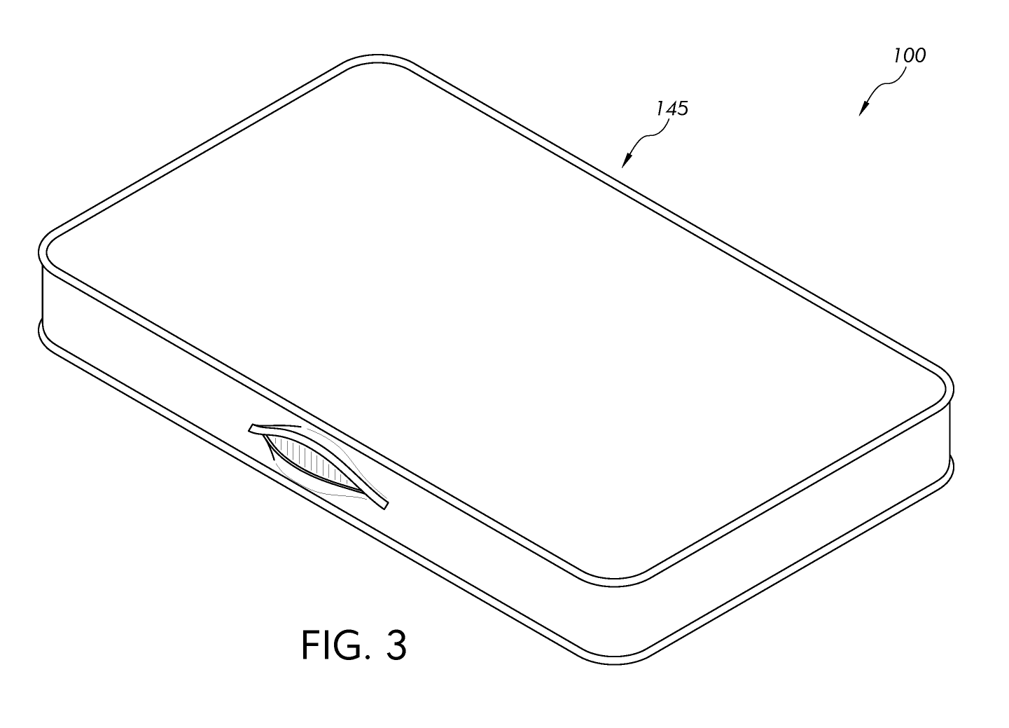

In some embodiments, the device for simulating a human presence 100 may include a mattress assembly 145. FIG. 3 is a perspective view of the mattress assembly 145 of FIG. 1, according to an embodiment of the present disclosure. Embodiments of mattress assembly 145 may include an infant crib mattress, a toddler-bed mattress, a full-size mattress, and/or a futon mattress. Embodiments of device for simulating a human presence 100 may also include a first-timer configured to terminate simulating the human presence of the remote person after a predetermined time interval. Similarly, some embodiments may include second-timer configured to begin simulating the human presence of the remote person after a predetermined time interval.

Returning to FIG. 1, according to one embodiment, the device for simulating a human presence 100 may be arranged as a kit 105. In particular, the device for simulating a human presence 100 may further include a set of instructions 155. The instructions 155 may detail functional relationships in relation to the structure of the device for simulating a human presence 100 (such that the device for simulating a human presence 100 can be used, maintained, or the like, in a preferred manner).

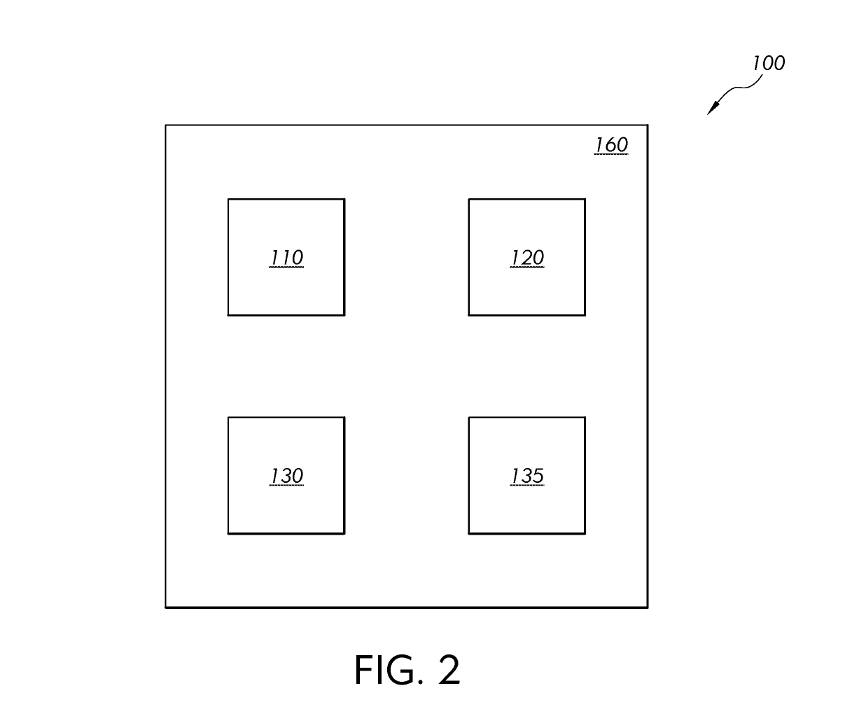

FIG. 2 diagrammatically shows the components of the device for simulating a human presence 100 of FIG. 1, according to an embodiment of the present disclosure. As illustrated, the device for simulating a human presence 100 may include a speaker 110, a heartbeat simulator 120, a processor 130, and a power supply 135. The device for simulating a human presence 100 may also include an external casing 160 which is impact resistant for increased safety and longevity during use, in some embodiments. One or more of the above components may be mounted to, embedded in, or otherwise housed in the external casing 160.

In embodiments, the speaker 110 may be configured to provide an audio output substantially similar to a voice of the remote person. The heartbeat simulator 120 may be configured to provide a tactile output onto a bed substantially similar to a heartbeat of the remote person. The processor 130 may be configured to drive the speaker 110 and the heartbeat simulator 120.

Further, the processor may be communicably 130 coupled to speaker 110 and to the heartbeat simulator 120. Similarly, the power supply 135 may be electrically coupled to the speaker 110 and to the heartbeat simulator 120, with the power supply 135 configured to power speaker 110 and heartbeat simulator 120. According to one embodiment, power supply 135 may include a DC battery. Further, the DC battery may be removable and replaceable and/or may be rechargeable. Alternately, power supply 135 may include an external AC electrical source.

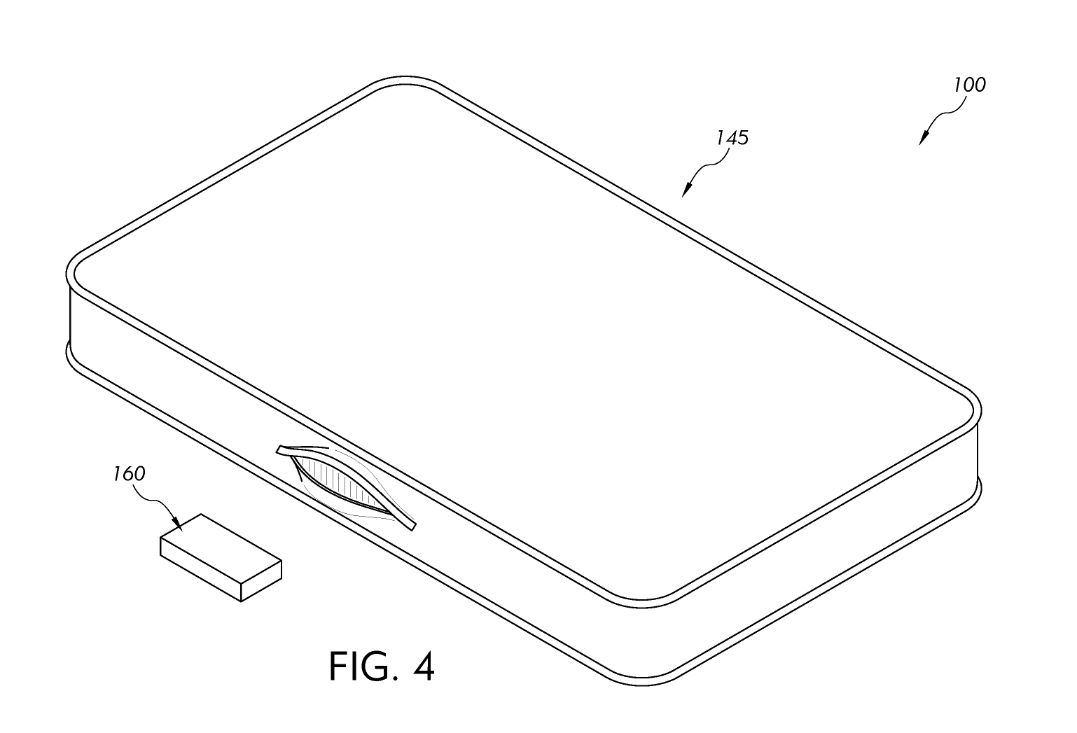

FIG. 4 is an exploded view of the device for simulating a human presence 100 of FIG. 1, according to an embodiment of the present disclosure. As shown, the speaker 110, the heartbeat simulator 120, the processor 130, and the power supply 135 may be removable from mattress assembly 145. Further, one or more of the above components and be conveniently embedded in external casing 160 such that they form one module that may be inserted and removed from mattress assembly 145 as a single electronic device.

In embodiments, device for simulating a human presence 100 may include the capability to record and playback human voice via the processor 130. Embodiments may also include the capability to download a pre-recorded human voice via the processor 130.

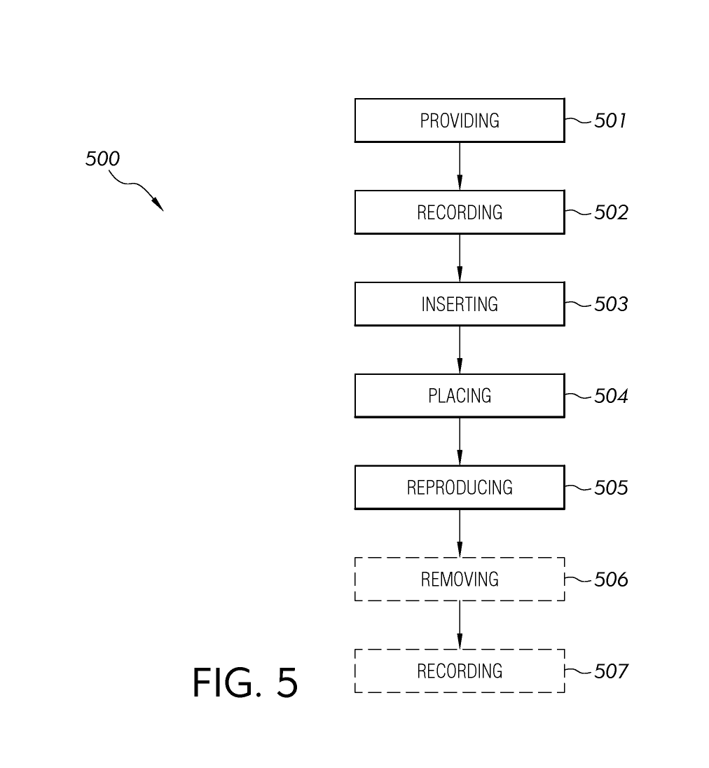

FIG. 5 is a flow diagram illustrating a method for method of using device for simulating a human presence 500, according to an embodiment of the present disclosure. In particular, the method of using a device for simulating a human presence 500 may include one or more components or features of the device for simulating a human presence 100 as described above. As illustrated, the method of using device for simulating a human presence 500 may include the steps of: step one 501, providing the device for simulating a human presence as an electronic device with a mattress assembly; step two 502, recording a first audio track on the device for simulating a human presence; step three 503, inserting the electronic device of the device for simulating a human presence into its mattress assembly; step four 504, placing a user (e.g., child, infant, etc.) upon mattress assembly; step five 505, reproducing the first audio track such that the user can hear the audio track; step six 506, removing the user from the mattress assembly; and step seven 507, recording a second audio track on device for simulating a human presence.

It should be noted that step six 506 and step seven 507 are optional steps and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for using a recordable mattress device (e.g., different step orders within above-mentioned list, elimination or addition of certain steps, including or excluding certain maintenance steps, etc.), are taught herein.

The embodiments of the disclosure described herein are exemplary and numerous modifications, variations and rearrangements can be readily envisioned to achieve substantially equivalent results, all of which are intended to be embraced within the spirit and scope of the disclosure. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for simulating a human presence of a remote person, the device comprising:
a speaker configured to provide an audio output substantially similar to a voice of the remote person;
a heartbeat simulator configured to provide a tactile output onto a bed substantially similar to a heartbeat of the remote person;
a processor communicably coupled to the speaker and to the heartbeat simulator, the processor configured to drive the speaker and the heartbeat simulator; and
a power supply electrically coupled to the speaker and to the heartbeat simulator, the power supply configured to power the speaker and the heartbeat simulator.

2. The device of claim 1, further including a first-timer configured to terminate simulating the human presence of the remote person after a predetermined time interval.

3. The device of claim 1, further comprising a second-timer configured to begin simulating the human presence of the remote person after a predetermined time interval.

4. The device of claim 1, further comprising a mattress assembly.

5. The device of claim 4, wherein the mattress assembly is an infant crib mattress.

6. The device of claim 4, wherein the mattress assembly is a toddler-bed mattress.

7. The device of claim 4, wherein the mattress assembly is a full-size mattress.

8. The device of claim 4, wherein the mattress assembly is a futon mattress.

9. The device of claim 4, wherein the speaker, the heartbeat simulator, the processor, and the power supply are removable from the mattress assembly.

10. The device of claim 1, wherein the power supply includes a DC battery.

11. The device of claim 10, wherein the DC battery is removable and replaceable.

12. The device of claim 10, wherein the DC battery is rechargeable.

13. The device of claim 1, wherein the power supply includes an external AC electrical source.

14. The device of claim 1, wherein the device is configured to record and playback human voice via the processor.

15. The device of claim 1, wherein the device is configured to download a pre-recorded human voice via the processor.

16. The device of claim 1, wherein the device includes an external casing that is impact resistant and configured for increased safety and longevity during use.

17. A device for simulating a human presence of a remote person, the device comprising:
a speaker configured to provide an audio output substantially similar to a voice of the remote person;
a heartbeat simulator configured to provide a tactile output onto a bed substantially similar to a heartbeat of the remote person;
a processor communicably coupled to the speaker and to the heartbeat simulator, the processor configured to drive the speaker and the heartbeat simulator;
a power supply electrically coupled to the speaker and to the heartbeat simulator, the power supply configured to power the speaker and the heartbeat simulator;
a first-timer configured to terminate simulating the human presence of the remote person after a predetermined time interval;
a second-timer configured to begin simulating the human presence of the remote person after a predetermined time interval; and
a mattress assembly; and
wherein the mattress assembly is an infant crib mattress;
wherein the speaker, the heartbeat simulator, the processor, and the power supply are removable from the mattress assembly;
wherein the power supply includes a DC battery;
wherein the DC battery is removable and replaceable;
wherein the DC battery is rechargeable;
wherein the device is configured to record and playback human voice via the processor;
wherein the device is configured to download a pre-recorded human voice via the processor; and
wherein the device includes an external casing that is impact resistant and is configured for increased safety and longevity during use.

18. The device of claim 17, further comprising set of instructions; and
wherein the device is arranged as a kit.

19. A method of using a device for simulating a human presence, the method comprising the steps of:
providing the device for simulating a human presence as an electronic device with a mattress assembly;
recording a first audio track on the device for simulating a human presence;
inserting the electronic device of the device for simulating a human presence into its mattress assembly;
placing a user upon the mattress assembly; and
reproducing the first audio track such that the user can hear the first audio track.

20. The method of claim 19, further comprising the steps of:
removing the user from the mattress assembly; and
recording a second audio track on the device for simulating a human presence.
